(19) United States
(12) Patent Application Publication
(10) Pub. No.: US 2001/0046504 A1
(43) Pub. Date: Nov. 29, 2001

(54) CYCLOPHOSPHAMIDE FILM-COATED TABLETS

(76) Inventors: JURGEN ENGEL, ALZENAU (DE); JURGEN RAWERT, ALZENAU (DE); DIETER SAUERBIER, WERTHER (DE); BURKHARD WICHERT, BIELEFELD (DE)

Correspondence Address: PILLSBURY MADISON & SUTRO LLP, INTELLECTUAL PROPERTY GROUP, 1100 NEW YORK AVENUE NW, NINTH FLOOR EAST TOWER, WASHINGTON, DC, 200053918

(21) Appl. No.: 09/333,256

(22) Filed: Jun. 15, 1999

(30) Foreign Application Priority Data
Jun. 15, 1998 (DE) 198 26 517.4

(51) Int. Cl. A61K 9/00

(52) U.S. Cl. 424/400

(57) ABSTRACT

1. [sic] The invention relates to film-coated tablets with cyclophosphamide as active compound, which in the core comprise cyclophosphamide, one or more fillers, one or more dry binders but no preswollen starch, flow regulators and lubricants.

According to a preferred embodiment of the invention, the core of the film-coated tablet comprises as a filler lactose monohydrate, D-mannitol or CaHPO₄, nonpreswollen cornstarch or microfine cellulose as a dry binder, highly disperse silica as a flow regulator and magnesium stearate, stearic acid, glycerol palmitostearate, polyethylene glycol, talc or glycerol monobeherate [sic] as a lubricant.

The invention relates to cyclophosphamide film-coated tablets and to a process for their preparation. The invention can be used in the pharmaceutical industry

Cyclophosphamide is an agent having a broad antitumor spectrum which has been introduced [sic] in chemotherapy for decades for the treatment of solid tumors such as mastocarcinoma, bronchial carcinoma and hemoblastoses.

Until now, on [sic] known pharmaceutical forms have been tablets, coated tablets and mainly lyophilizates with various auxiliaries such as mannitol or urea.

EP 0519099 describes tablets comprising cyclophosphamide and preswollen starch, prepared by a direct tableting process.

Since cyclophosphamide is dangerous to health and for this reason direct contact with this substance represents a potential risk, the tablets prepared according to EP 0519099 are used as cores for press-coated tablets and thus coated by means of a second tableting. This process is technically complicated. Special tableting machines are furthermore needed for the preparation of press-coated tablets.

The need thus exists for a simple and economical preparation of solid pharmaceutical form [sic] comprising cyclophosphamide for oral administration.

It is necessary to take into consideration here that the pharmaceutical forms have to be coated in order that direct contact with the cytotoxic active compound is avoided.

It is moreover known that cyclophosphamide is chemically labile, thus the stability of the pharmaceutical forms must also be taken into consideration.

Surprisingly, it has been possible to prepare film-coated tablets comprising cyclophosphamide without the use of preswollen starch.

Suitable auxiliaries were selected on the basis of the compatibility investigations mentioned in Example I [sic] . It was surprising in this context that the stability of cyclophosphamide is somewhat indifferent in the presence of preswollen starch.

It was moreover surprising that the finished film-coated tablets exhibit an adequate stability although the active compound, due to the preparation, is stressed during the film-coating process by moisture and heat.

### EXAMPLE 1

Investigations on the Compatibility of Cyclophosphamide with Various Tableting Auxiliaries

53.5 mg of cyclophosphamide and 86.5 mg of (auxiliary 1-10) [sic] or 3.0 mg of (auxiliary 11-18) [sic] were in each case mixed and compressed. The pressed tablets were stored at 31° C. for 6 months. The decomposition of the active compound was carried out [sic] by means of chloride determination.

The results are summarized in the following table.

| | | | Decomposition | Dis- |
|---|---|---|---|---|
| Function of the | | | of cyclo- | colora- |
| auxiliary | | Auxiliary | phosphamide | tion |
| FILLER | 1 | Lactose, anhydrous | 2.52 | ++ |
| | 2 | Calcium phosphate | 3.85 | − |
| | 3 | Calcium phosphate | 2.02 | − |
| | | anhydrous | | |
| | 4 | Emcompress | 1.50 | |
| | | (CaHPO₄) | | |
| | 5 | D-mannitol | 1.15 | − |
| | 6 | Lactose | 0.70 | − |
| | | monohydrate | | |
| FILLER/DRY | 7 | Microcrystalline | 1.50-1.73* | − |
| BINDER/ | | cellulose | | |
| DISINTEGRATION | 8 | Cellulose (Elcema) | 0.85-1.32* | −+ |
| PROMOTER | 9 | Preswollen starch | 1.02 | −+ |
| | 10 | Cornstarch | 0.75 | − |
| DISINTEGRATION | 11 | Crosslinked poly- | 1.5 | ++ |
| PROMOTER | | vinyl pyrrolidone | | |
| FLOW | 12 | Highly disperse | 0.46-1.72* | −+ |
| REGULATOR | | silica | | |
| FLOW | 13 | Magnesium stearate | 1.51 | −+ |
| REGULATOR/ | 14 | Stearic acid | 0.94 | −+ |
| LUBRICANT | 15 | Glycerol | 0.82 | − |
| | | palmitostearate | | |
| | 16 | Polyethylene | 0.68 | − |
| | | glycol | | |
| | 17 | Talc | 0.55 | − |
| | 18 | Glycerol | 0.30 | − |
| | | monobeherate [sic] | | |

*Dependent on type

### EXAMPLE 2

Preparation of Tablet Cores (50 mg of Cyclophosphamide)

Direct Tableting

0.535 mg of cyclophosphamide, 0.390 mg of lactose monohydrate, 0.400 mg of microfine cellulose, 0.200 mg of cornstarch, 0.040 mg of talc and 0.020 mg of highly disperse silica are sieved and homogenized. 0.015 mg of magnesium stearate is then added and mixed. The mass prepared in this way is processed to give tablets:

| | |
|---|---|
| Weight: | 160 mg |
| Hardness: | >30N |
| Disintegration: | <10 min. |

### EXAMPLE 3

Preparation of Film-coated Tablets (50 mg of Cyclophosphamide)

11.83 g of polyethylene glycol and 2.37 g of polysorbate 80 are dissolved in 75.21 g of water.

1.9 g of carboxymethylcellulose sodium are dissolved in 80.0 g of water. The solutions are brought together. 23.67 g of talc, 23.67 g of titanium dioxide and 0.24 g of simeticone [sic] are then added and the mixture is homogenized. 17.73 g of a 30% strength ethyl acrylate/methyl metharcrylate [sic] copolymer dispersion in water are then added. The tablet cores are then sprayed with the prepared suspension in a suitable apparatus:

Theoretical weight of a film-coated tablet: 166 mg

### EXAMPLE 4

Investigation of the Stability of Cyclophosphamide Film-coated Tablets

Decomposition of cyclophosphamide after 3 months

| | 26° C./60% RH | 31° C./40% |
|---|---|---|
| Batch 1 | 0.30 | 4.12 |
| Batch 2 | 0.17 | 2.36 |

Stability of the film-coated tablets of up to 3 years is expected on storage at <25° C.

1. A film-coated tablet with cyclophosphamide as active compound, comprising in the core cyclophosphamide, one or more fillers, one or more dry binders but no preswollen starch, flow regulators and lubricants.

2. The film-coated tablet as claimed in claim 1, comprising in the core as a filler lactose monohydrate, D-mannitol or CaHPO₄, nonpreswollen cornstarch or microfine cellulose as a dry binder, highly disperse silica as a flow regulator and magnesium stearate, stearic acid, glycerol palmitostearate, polyethylene glycol, talc or glycerol monobeherate [sic] as a lubricant.

3. The film-coated tablet as claimed in claim 2, where the cores can comprise the auxiliaries either individually or alternatively in any desired mixture.

4. The film-coated tablet as claimed in claims 1 to 3, comprising, per part of cyclophosphamide in the core, lactose monohydrate, microfine cellulose, nonpreswollen cornstarch, talc, highly disperse silica and magnesium stearate in the following ratio:
lactose monohydrate 0.2-1.5, preferably 0.5-1, particularly 0.73
microfine cellulose 0.2-1.5, preferably 0.5-1, particularly 0.74
nonpreswollen cornstarch 0.1-1.5, preferably 0.2-0. 7, particularly 0.37
talc 0.01-1.5, preferably 0.05-0.08, particularly 0.07
highly disperse silica 0.01-0.1, preferably 0.01-0.5, particularly 0.04
magnesium stearate 0.01-0.1, preferably 0.01-0.05, particularly 0.03.
